(19) United States
(12) Patent Application Publication
(10) Pub. No.: US 2009/0163897 A1
(43) Pub. Date: Jun. 25, 2009

(54) Illuminated Ophthalmic Instruments

(76) Inventor: Allen W. Skinner, Chesterfield, MO (US)

Correspondence Address: Bausch & Lomb Incorporated, One Bausch & Lomb Place, Rochester, NY, 14604-2701, US

(21) Appl. No.: 11/960,038

(22) Filed: Dec. 19, 2007

(51) Int. Cl. A61B 18/18; A61M 1/00

(52) U.S. Cl. 606/4; 604/319

(57) ABSTRACT

An ophthalmic instrument 10 for providing illumination to a surgical site, includes a housing 12 defining an internal volume 14, at least one pathway 16 extending through the internal volume of the housing to external of the instrument that is designed for irrigation or aspiration during surgery, and a fiber optic component 18 positioned within the at least one pathway 16 for providing illumination to a surgical site. A modified aspiration tube 300, includes aspiration tubing 350 with an internal volume 360 and a fiber optic component 318 spliced within internal volume 360.

### BACKGROUND OF THE INVENTION

1. Field of the Invention

This invention relates specifically to the field of ophthalmic surgical instruments. More particularly, the present application is directed to the incorporation of a fiber optic component into an ophthalmic instrument for providing illumination to a surgical site.

2. Description of Related Art

During ophthalmic surgery, it is important to have light inside a patient's eye so that the posterior portion of the eye is illuminated enough for the surgeon to visualize a surgical site.

Various illumination schemes for posterior surgery exist, as the operating room lights are usually off. Often a device is placed inside the eye that has to be controlled separately from the instrument being used by the surgeon. There are also devices that are affixed to the eye that provide an illuminated port for the ophthalmic instruments to pass through into the eye.

It is also known to provide illumination to surgical instruments through fiber optics incorporated into the handpiece. However, these instruments only provide illumination adjacent the proximal end of the tip of the surgical instrument to be inserted into the eye and require a specially designed instrument to accommodate the fiber optic.

There exists the need for an improved ophthalmic instrument for providing illumination to the surgical site.

### BRIEF DESCRIPTION OF FIGURES

FIG. 1. is a cut-away graphical view of an ophthalmic instrument, in accordance with the present invention;

FIG. 2 is a cut-away view of a vitrectomy probe which has been modified, in accordance with the present invention;

FIG. 3 is a cut-away view of an ultrasonic handpiece which has been modified, in accordance with the present invention;

FIG. 4 is a cut-away view of a portion of the ultrasonic handpiece illustrated in FIG. 3; and

FIG. 5 is a cut-away graphical view of an aspiration tube with a fiber optic component spliced within an internal volume of the tube.

### DETAILED DESCRIPTION

The present invention incorporates a light source directly into an ophthalmic surgical instrument so that a surgeon can easily illuminate a surgical site within the eye.

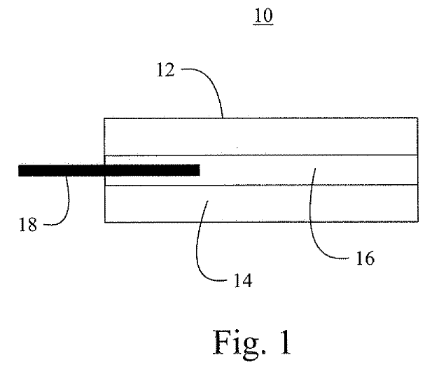

The present invention provides an ophthalmic instrument for providing illumination to a surgical site. FIG. 1 shows a cut-away graphical representation of an ophthalmic surgical instrument 10, in accordance with the present invention. Ophthalmic instrument 10 includes a housing 12, defining an internal volume 14, at least one pathway 16 extending through internal volume 14 of housing 12 to external of ophthalmic instrument 10 and a fiber optic component 18. Internal pathway 16 has a function in communication with a surgical site other than accommodating the fiber optic 18, such as an aspiration pathway or an irrigation pathway, both of which are commonly found in ophthalmic surgical instruments.

Fiber optic component 18 is positioned within the at least one pathway 16 in a way that provides illumination to a surgical site. An optical fiber may be a glass or plastic fiber designed to transmit light along its length. Light is kept in the core of the optical fiber by internal reflection. Optical fibers are often long, thin strands arranged in bundles called optical cables. Such fibers are widely used in illumination applications where bright light needs to be shone on a target without a clear line-of-sight path.

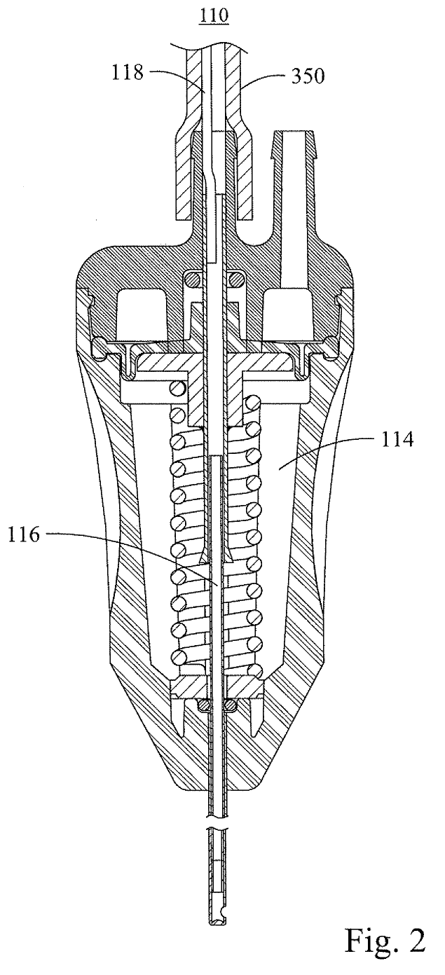

FIGS. 2-4 show cross sections of known ophthalmic surgical instruments which have been modified according to the present invention.

In one embodiment, FIG. 2 shows a cross section of an instrument, specifically a vitrectomy probe 110, which includes a housing 112, defining an internal volume 114 and an internal pathway 116 which extends through internal volume 114 to external of vitrectomy probe 110. In some embodiments, internal pathway 116 functions as an aspiration pathway. A fiber optic component 118 is positioned within internal pathway 116 in a way that provides illumination to a surgical site, but yet does not interfere with the aspiration function of pathway 116. FIG. 2 shows a fiber optic component 118 spliced within an aspiration tube 350 which will be described in greater detail hereinafter. In alternative embodiments, fiber optic component 118 does not necessarily have to be positioned within aspiration tube 350, but could instead be directly positioned within internal pathway 116 by inserting fiber optic component 118 through housing 112 and into internal pathway 116.

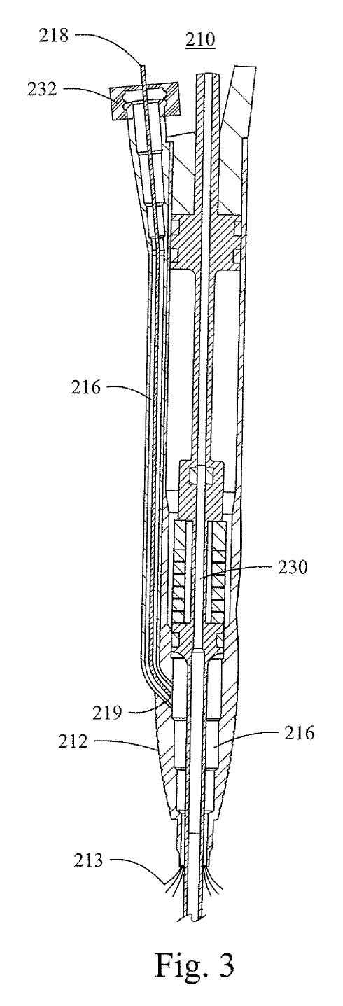

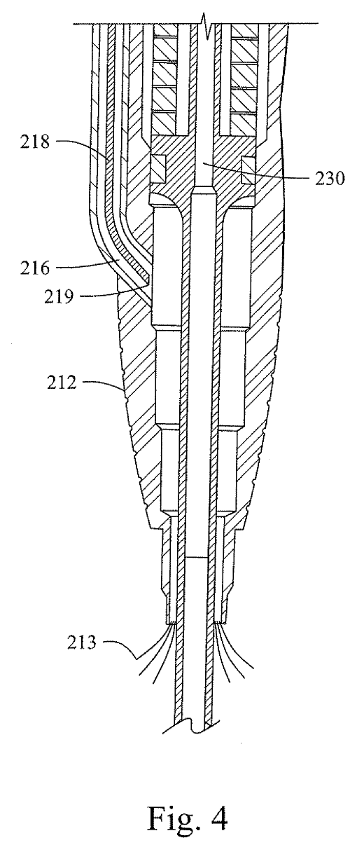

In another embodiment, FIGS. 3 and 4 show a cross section of an instrument, specifically an ultrasonic handpiece 210, which includes a housing 212, defining an internal volume and an internal pathway 216. Internal pathway 216 functions as an irrigation pathway when handpiece 210 is used in standard phacoemulsification surgery, but when handpiece 210 is used as a fragmentation device in vitreoretinal surgery, pathway 216 functions to direct a fiber optic towards the distal end of handpiece 210, as shown. A fiber optic component 218 is positioned within internal pathway 216 in a way that provides illumination to a surgical site. FIG. 3 shows fiber optic component 218 being held in place by luer 232. The internal volume 216 is typically a polished metal surface when used for irrigation. The polished metal surface acts as a good light reflector to direct light out of handpiece 210, as indicated by lines 213. Fiber optic component 218 has a length of fiber optic so that it may be connected to an illumination source (not shown). The luer 232 connects the fiber optic to handpiece 210. The luer 232 is positioned on the length of fiber optic so that a distal end 219 of the fiber optic component 218 is properly positioned within handpiece 210 to provide lighting to a surgical site.

Ultrasonic handpiece 210 also contains a second internal pathway 230. In some embodiments, this second internal pathway 230 functions as an aspiration pathway. Therefore, fiber optic component 218 could also be positioned within second internal pathway 230 in a way that provides illumination to a surgical site, similar to the embodiment shown in FIG. 2. As described above, if fiber optic 218 is placed within aspiration pathway 230, it is important that fiber optic 218 not interfere with the aspiration function.

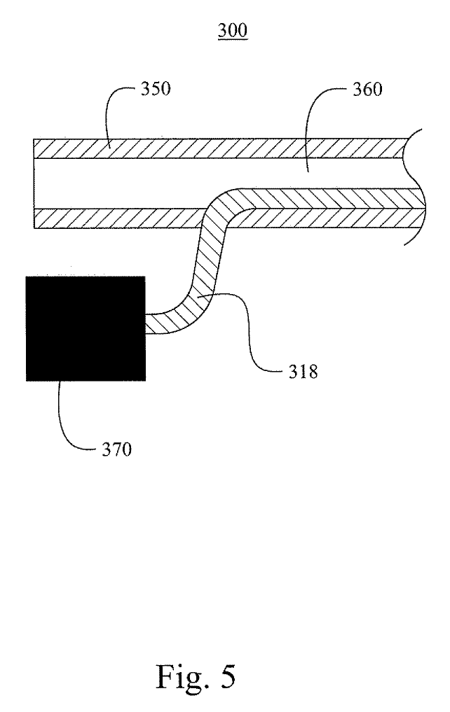

The present invention further provides a modified aspiration tube comprising a fiber optic component 318. FIG. 5 shows a cut-away graphical representation of a modified aspiration tube 300. Modified aspiration tube 300 comprises aspiration tubing 350, which includes an internal volume 360. A fiber optic component 318 is spliced within internal volume 360. FIG. 5 shows a light pipe connector 370 connected to fiber optic component 318. Obviously, it is important that the splice be fluidly sealed, so that tubing 350 does not leak during surgery. Such a seal can be made by any known means, such as adhesives, gaskets, or other sealing means. It is also noted that all the fiber optics described above will be connected to a light source that has not been shown.

By using existing pathways within known surgical instruments a fiber optic is able to provide much needed illumination to a surgical site without the need for a specially designed handpiece. This allows surgeons to use instruments they are familiar with that can be retrofitted to provide illumination and does not require the purchase of a new instruments.

We claim:

1. An ophthalmic instrument for providing illumination to a surgical site, the ophthalmic instrument comprising:
a housing, defining an internal volume;
at least one pathway extending through the internal volume of the housing to external of the instrument that is designed for irrigation or aspiration during surgery; and
a fiber optic component positioned within the at least one pathway for providing illumination to a surgical site.

2. The ophthalmic instrument of claim 1, wherein the instrument is an ultrasonic handpiece.

3. The ophthalmic instrument of claim 1, wherein the instrument is a vitrectomy probe.

4. The ultrasonic handpiece of claim 2, wherein the fiber optic component is positioned within the irrigation pathway.

5. The ultrasonic handpiece of claim 2, wherein the fiber optic component is positioned within the aspiration pathway.

6. The vitrectomy probe of claim 3, wherein the fiber optic component is positioned within the aspiration pathway.

7. A modified aspiration tube comprising:
an internal volume; and
a fiber optic spliced within the internal volume of the tube.

8. A fiber optic component comprising:
a length of fiber optic;
a luer for connection of the fiber optic to a surgical handpiece; and
wherein the luer is positioned on the length of fiber optic so that a distal end of the fiber optic is properly positioned within the surgical handpiece to provide lighting to a surgical site.
